(19) United States
(12) Patent Application Publication
(10) Pub. No.: US 2021/0113097 A1
(43) Pub. Date: Apr. 22, 2021

(54) HEART FAILURE DEGREE-OF-EXACERBATION DETERMINATION SYSTEM AND HEART FAILURE DEGREE-OF-EXACERBATION DETERMINATION METHOD

(71) Applicant: NEC Corporation, Minato-ku, Tokyo (JP)

(72) Inventors: Yuji OHNO, Tokyo (JP); Masahiro KUBO, Tokyo (JP); Katsumi ABE, Tokyo (JP); Ersin ALTINTAS, Turkey (TR); Takeshi AKAGAWA, Tokyo (JP); Tetsuri ARIYAMA, Tokyo (JP); Hiroaki SHIMOKAWA, Miyagi (JP); Yasuhiko SAKATA, Miyagi (JP); Ryuji TUBURAYA, Miyagi (JP)

(73) Assignee: NEC Corporation, Minato-ku, Tokyo (JP)

(21) Appl. No.: 17/040,298

(22) PCT Filed: Feb. 18, 2019
(86) PCT No.: PCT/JP2019/005835

§ 371 Date: Sep. 22, 2020

(30) Foreign Application Priority Data
Apr. 3, 2018 (JP) 2018-071734

(51) Int. Cl. A61B 5/0205; A61B 5/00; A61B 5/02

(52) U.S. Cl. CPC A61B 5/0205; A61B 5/4842; A61B 5/443; A61B 5/024; A61B 5/444; A61B 5/02028; A61B 5/442

(57) ABSTRACT

This heart failure degree-of-exacerbation determination system comprises a storage device (11) and an arithmetic device (12). The arithmetic device (12) includes a heart failure degree-of-exacerbation determination means (121) for determining the degree-of-exacerbation of heart failure, on the basis of stored information which is stored in the storage device (11) and the correlation among a plurality of evaluation values which are associated with a acral portion of a patient.

### TECHNICAL FIELD

The present invention relates to a heart failure degree-of-exacerbation determination system and a heart failure degree-of-exacerbation determination method for determining the degree-of-exacerbation of a patient's heart failure.

### BACKGROUND ART

Heart failure is the largest cause of death in the world. In the past, there were a large number of deaths caused by acute myocardial infarction, but this is not an increasing trend due to recent advances in treatment. On the other hand, deaths due to heart failure have been increasing. Heart failure leads to frequent and repeated hospitalization, and the costs of medical treatment is also a major problem. Therefore, early detection of heart failure is an important challenge as well as determining its degree-of-exacerbation.

Acute heart failure is defined as “a condition in which an organic and/or functional abnormality in the heart causes a rapid collapse of the compensatory turning point of the heart pump function, causing an increase in the end-ventricular diastolic pressure and a perfusion failure to the major organs, resulting in an abrupt appearance or deterioration of the symptom or sign based on it.” Chronic heart failure is defined as “a condition in which the pumping function of the heart is lowered by the chronic myocardial damage, and the volume of blood enough to meet the oxygen demand of the peripheral major organs cannot be discharged absolutely or relatively, causing congestion in the lungs, vein systems, or both systems and causing damage to daily life.” By detecting these conditions, devices and systems that evaluate whether or not a heart failure has occurred have been developed. For example, Patent Documents 1 to 4 describe an apparatus and a system for evaluating heart failure.

In the multiple sensor scheme for heart failure patient management described in Patent Document 1, at least one sensor provides a sensor signal including physiological information with a plurality of implantable sensors, and a first rule is used to determine whether or not a detected physiological change event indicates a change in the heart failure state of a subject, and a second rule is used to determine whether or not a heart failure determination in the first rule is invalidated, and to indicate whether or not a change has occurred in a heart failure state according to the first and second rules.

The apparatus and system for detecting and evaluating heart failure described in Patent Document 2 monitors a patient with a device comprising: a patient interface; a non-invasive sensor for generating signals associated with the characteristics of the patient's respiration; and a processor, which is coupled to the sensor, configured to control and store a determination of a heart failure symptom change indicator based on the measured signal, to compare the indicator with a pre-stored and determined value of the indicator from one or more prior treatment sessions and to determine a risk of an event of a heart failure compensatory failure occurring in the patient; and a risk of occurrence of an event of a heart failure compensatory failure.

The detection of electrical and mechanical cardiovascular activity described in Patent Document 3 has a transmitter for transmitting electromagnetic signals with a predetermined frequency to the patient's chest, a receiver for receiving reflected and Doppler frequency-shifted electromagnetic waves, and an ECG unit for capturing ECG signals of the patient's heart, to detect electrical and mechanical cardiovascular activities of the patient, particularly premature compensatory failure of a congestive heart failure patient.

Patent Document 4 describes a portable automatic monitoring of a congestive heart failure patient, which focuses on monitoring the heart and respiration.

### PRIOR ART DOCUMENTS

### Patent Document

[Patent Document 1] Japanese Patent No. 5300982

[Patent Document 2] Japanese Patent No. 5443875

[Patent Document 3] JP 2010-540148 A

[Patent Document 4] JP 2009-540953 A

### SUMMARY OF THE INVENTION

### Problem to be Solved by the Invention

However, as regards the device, system and scheme described in Patent Documents 1 to 4, there are the following problems.

In the multiple sensor system for heart failure patient management described in Patent Document 1, it relates to an implantable medical device such as a pacemaker and an implantable cardioverter, invasive and patient burden is large.

In the device and system for detecting and evaluating heart failure described in Patent Document 2, changes or exacerbations of patient symptom are determined by the increased number and/or duration of apnea, respiration depression, and/or chain Stokes respiration. However, large equipment is required to measure the characteristics of respiration, and routine evaluation is difficult.

In the detection of electrical and mechanical cardiovascular activity described in Patent Document 3, for more reliable evaluation, it is necessary to continue to place the device in the same position of the body. However, in evaluating daily changes in cardiovascular activity, it is difficult for a person who lacks knowledge and experience to place the device in the same position of the body every day, and it is difficult to sustain high reliability.

In the monitoring method described in Patent Document 4, it is necessary to take continuous measurements for a long time because periodic variable respiration is required, the effects of artifacts are large when the patient takes various postures and actions during that time, and therefore, significant limitations are placed on obtaining reliable measurement.

Thus, in the apparatus and system described in Patent Documents 1 to 4, because the sensor is implantable and because the burden that is placed on the patient is large, it is difficult to conduct large scale routine evaluations that provide high reliability due to misalignment, and the problem occurs in which the measurement time is long.

An object of the present invention is to provide a heart failure degree-of-exacerbation determination system and a heart failure degree-of-exacerbation determination method for solving the above problems.

### Method for Solving the Problem

A heart failure degree-of-exacerbation determination system of the present invention, comprising:

a storage device; and

an arithmetic device,

wherein the arithmetic device has a heart failure degree-of-exacerbation determination means that determines the degree-of-exacerbation of heart failure, based on the stored information stored in the storage device and based on the correlation between a plurality of evaluation values associated with the acral portion of the patient.

Further, the heart failure degree-of-exacerbation determination method of the present invention determines the degree-of-exacerbation of heart failure based on information and correlations between multiple evaluation values related to the patient's acral portion.

### Effect of the Invention

As described above, according to the present invention, the evaluation value can be acquired by a non-invasive sensor and the burden on the patient is light; evaluations can be routinely and portably carried out; it is possible to avoid the problem of positional deviation; measurements can be undertaken in a short period.

### BRIEF DESCRIPTION OF THE DRAWINGS

FIG. 1 It is a block diagram illustrating a first embodiment of the heart failure degree-of-exacerbation determination system of the present invention.

FIG. 2 It is a diagram illustrating an example of the configuration of the information shown in FIG. 1.

FIG. 3 It is a flowchart for explaining a heart failure degree-of-exacerbation determination method in the heart failure degree-of-exacerbation determination system shown in FIG. 1.

FIG. 4 It is a block diagram illustrating a second embodiment of the heart failure degree-of-exacerbation determination system of the present invention.

FIG. 5 It is a flowchart for explaining a heart failure degree-of-exacerbation determination method in the heart failure degree-of-exacerbation determination system shown in FIG. 4.

FIG. 6 It is a block diagram illustrating a third embodiment of the heart failure degree-of-exacerbation determination system of the present invention.

FIG. 7 It is a flowchart for explaining a heart failure degree-of-exacerbation determination method in the heart failure degree-of-exacerbation determination system shown in FIG. 6.

FIG. 8a It is a diagram showing an example of a statistically created deterioration degree-of-exacerbation map.

FIG. 8b It is a diagram showing an example of a degree-of-exacerbation map created for each individual.

FIG. 9 Is a diagram showing an example of a degree-of-exacerbation map expressed in three dimensions.

FIG. 10 It is a block diagram illustrating a fourth embodiment of the heart failure degree-of-exacerbation determination system of the present invention.

FIG. 11 It is a flowchart for explaining a heart failure degree-of-exacerbation determination method in the heart failure degree-of-exacerbation determination system shown in FIG. 10.

FIG. 12 It is a block diagram illustrating a fifth embodiment of the heart failure degree-of-exacerbation determining system of the present invention.

FIG. 13 It is a flowchart for explaining a heart failure degree-of-exacerbation determination method in the heart failure degree-of-exacerbation determination system shown in FIG. 12.

FIG. 14 It is a block diagram illustrating a sixth embodiment of the heart failure degree-of-exacerbation determination system of the present invention.

FIG. 15 It is a diagram schematically illustrating the posture of the patient.

### FORMS FOR IMPLEMENTING THE PRESENT INVENTION

Next, embodiments of the present invention will be described with reference to the accompanying drawings.

### First Embodiment

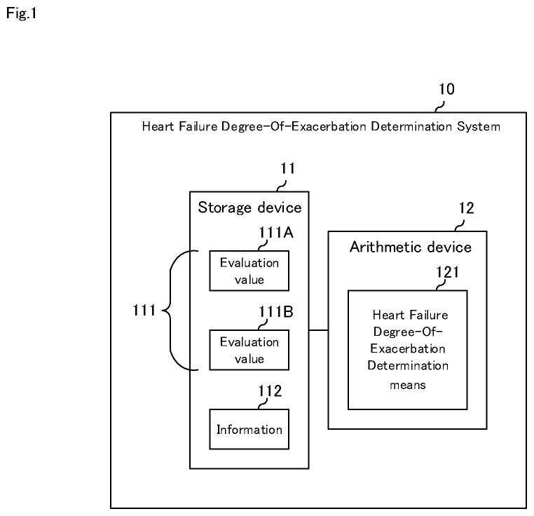

FIG. 1 is a block diagram illustrating a first embodiment of the heart failure degree-of-exacerbation determination system of the present invention; Referring to FIG. 1, heart failure degree-of-exacerbation determination system 10 according to a first embodiment of the present invention includes storage device 11, and arithmetic device 12.

Storage device 11 is a recording medium capable of reading and writing data, an HDD (Hard Disc Drive), a solid-state memory, or the like. Storage device 11 stores evaluation value 111 and information 112. Further, it may include a program or the like for executing heart failure degree-of-exacerbation determination means 121 to be described later.

Evaluation value 111 is a multiple evaluation value related to heart failure that can be acquired at the acral portion of the patient. As an example, FIG. 1 shows a case where two evaluation values 111 are used, i.e., evaluation value 111A and evaluation value 111B, but three or more evaluation values may be used. Evaluation value 111 is a value related to heart failure, which includes any of the following: congestion degree indicating the degree of congestion at the acral portion, hypoperfusion degree indicating the degree of hypoperfusion at the acral portion, pulse wave shape, cardiac output, heart rate, and respiration rate, and is acquired with a non-invasive sensor located at the acral portion.

The acral portion is near the end of the patient's body, for example, the hand (including the wrist, palm of the hand, finger of the hand, and back of the hand). Also, as another example, the acral portion is an foot (including the ankle, toe of the foot, sole of the foot, instep, and heel).

Congestion refers to a condition in which the flow of blood in the veins and capillary vessels in the organ tissues is stagnant at the end and increases because of heart disease (especially in the case of systemic congestion). During the onset of the left heart failure, the lowering of blood pressure by the lowering of the cardiac output and pulmonary congestion by the rise of the left atrial pressure occur in addition to the lowering of the blood flow of various organs. On the other hand, during the onset of the right heart failure, congestion of the venous system is the main cause, and excessive accumulation of fluid occurs in the whole body, especially in the lower extremities, resulting in leg edema as a sign of heart failure. In addition, it causes the symptom which reflects the poor circulation such as ascites, hepatomegaly, vein distension, etc.

Hypoperfusion refers to a state in which blood is not sufficiently spread to each organ and periphery due to a decrease in cardiac function. Symptoms such as whole-body malaise, easy fatigue, decrease in urine volume, feeling of extremities cold, etc. are caused.

Pulse wave shape, cardiac output, and heart rate reflect the condition of the heart, thus creating a relationship with heart failure. Respiratory rate is associated with heart failure as a symptom of pulmonary congestion. Here, the respiratory rate can be acquired by analyzing the pulse wave shape measured by a non-invasive sensor located at the acral portion.

Evaluation value 111 does not necessarily mean that there is a correlation with heart failure for values associated with single heart failure. For example, hypoperfusion alone does not distinguish coldness from heart failure. However, in the correlation of the values related to multiple heart failure among the evaluation values 111, since the state of heart failure can be estimated from different symptoms, it is more likely to reflect the degree-of-exacerbation of heart failure. Thus, among congestion and hypoperfusion, cardiac output, heart rate, and respiratory rate, any multiple correlations may reflect the state of cardiac function, the degree-of-exacerbation of heart failure, that is, the degree-of-exacerbation of heart failure.

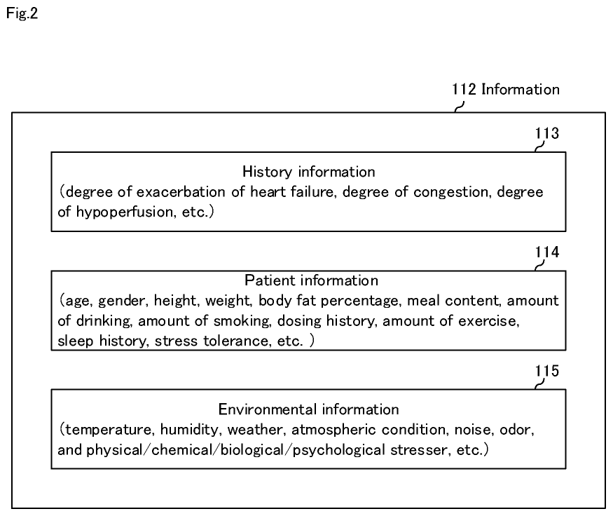

FIG. 2 is a diagram illustrating an example of the configuration of information 112 shown in FIG. 1. As shown in FIG. 2, information 112 is stored information including any of history information 113, patient information 114, and environmental information 115.

History information 113 is information such as the history of the determination result of the heart failure degree-of-exacerbation of the patient, the acquisition history of evaluation value 111. Patient information 114 is information such as age, gender, height, weight, body fat percentage, meal content, amount of drinking, amount of smoking, dosing history, amount of exercise, sleep history, stress tolerance, etc. related to the patient. Environmental information 115 is information related to the ambient environment of patient 1 such as temperature, humidity, weather, atmospheric condition, noise, odor, and physical/chemical/biological/psychological stress of the surrounding area.

Evaluation value 111 and information 112 need not necessarily be stored in the same recording medium, or storage device 11 may be configured by a plurality of recording media.

Arithmetic device 12 is a computer device that operates in accordance with a program, a CPU (Central Processing Unit), or the like. Heart failure degree-of-exacerbation determination means 121 is carried out according to the program, and the degree-of-exacerbation of heart failure is judged. The program may be provided via a communications network (e.g., the Internet) or may be provided by a computer readable recording medium. The computer readable recording medium is, for example, an optical disk such as a CD (Compact Disc) or a DVD (Digital Versatile Disc), a USB (Universal Serial Bus) solid state memory, a memory card, or the like. The storage medium may be storage device 11 or may be another device (not shown).

Hereinafter, the operation of heart failure degree-of-exacerbation determination system 10 will be specifically described.

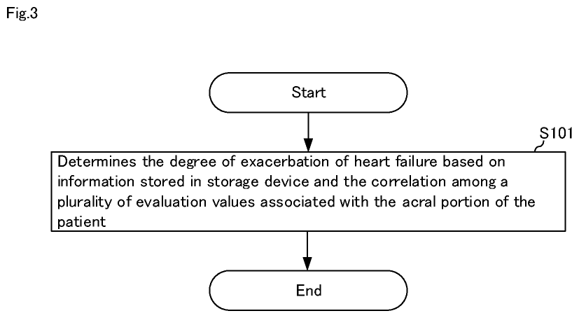

FIG. 3 is a flowchart for explaining a heart failure degree-of-exacerbation determination method in heart failure degree-of-exacerbation determination system 10 shown in FIG. 1. Hereinafter, the heart failure degree-of-exacerbation determination method will be described with reference to FIGS. 1 to 3.

In step S101, heart failure degree-of-exacerbation determination means 121 determines the degree-of-exacerbation of heart failure based on information 112 stored in storage device 11 and the correlation among a plurality of evaluation values 111 associated with the acral portion of the patient.

Information 112 may be acquired by a sensor that measures the patient or the surrounding environment and information 112 may be automatically stored in storage device 11, or information 112 may be stored in storage device 11 by the patient himself/herself, a person related to the patient, or a health care worker via the input device.

Evaluation value 111 may be acquired by a non-invasive type sensor unit disposed at the acral portion of the patient and automatically stored in storage device 11, or the value acquired by the sensor unit by the patient himself/herself, a person related to the patient, or a health care worker via the input device or a value determined by a physician or the like may be stored in storage device 11.

Heart failure degree-of-exacerbation determination means 121 judges the degree-of-exacerbation of heart failure from evaluation value 111 which reflects the state of the cardiac function based on history information 113, patient information 114, environment information 115.

The heart failure degree-of-exacerbation determination system of the present embodiment, is constituted by storage device 11 and arithmetic unit 12, determines the degree-of-exacerbation of heart failure using a plurality of evaluation values that can be acquired by a non-invasive type sensor disposed at the acral portion of the patient. Thus, it is a non-invasive type and the burden on the patient is light, it is portable and can be used for routine evaluations, and the occurrence of the problem of the positional deviation can be avoided, and measurements can be taken in a short time.

### Second Embodiment

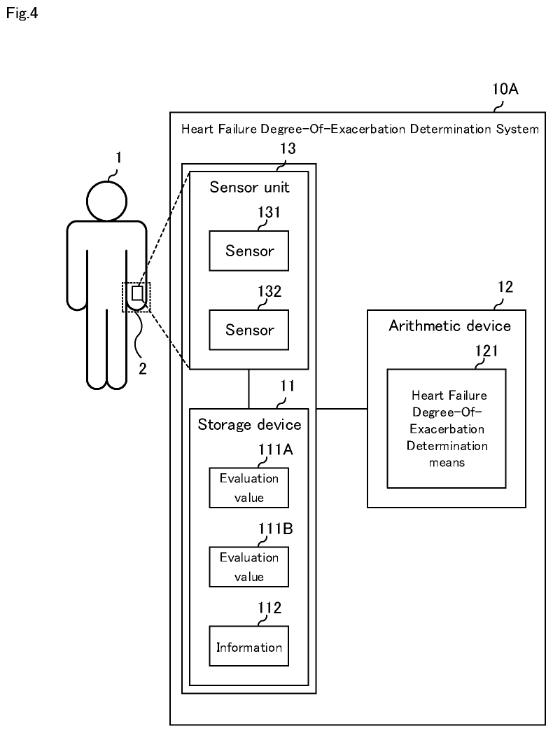

FIG. 4 is a block diagram illustrating a second embodiment of the heart failure degree-of-exacerbation determination system of the present invention. As shown in FIG. 4, the heart failure degree-of-exacerbation determination system according to the second embodiment of the present invention has the same configuration as that of the first embodiment, it further includes sensor unit 13. Here, mainly configuration different from the first embodiment will be described, a description of the same configuration will be omitted.

Heart failure degree-of-exacerbation determination system 10A according to a second embodiment of the present invention includes storage device 11, arithmetic device 12, and sensor unit 13, as shown in FIG. 4.

Sensor unit 13 includes one or more sensors and is located at acral portion 2 of the patient 1 shown in FIG. 4.

FIG. 4 schematically shows an example in which sensor unit 13 composed of two sensors (sensor 131 and sensor 132) is placed in the hand as the acral portion 2. Sensor unit 13 may be composed of one sensor or three or more sensors.

Sensor 131 and sensor 132 are non-invasive sensors capable of acquiring patient data, and include, for example, capacitive sensors, thermistors, magnetic sensors, laser light sensors (laser light sources and photodetectors), ultrasonic sensors (ultrasonic transmitters and receivers), image sensors (light sources and cameras), spectral sensors (light sources and optical spectral detectors). One or more types of sensors are appropriately selected to suit the ambient environment, the patient, and the required accuracy, and constitute sensor unit 13. Sensor 131 and sensor 132 may be non-invasive, either contact or non-contact. Whether to bring sensor 131 and sensor 132 into contact with acral portion 2 is appropriately selected depending on the type of the sensor and the characteristics of the sensor. The sensor unit 13 may include a power supply and a communication unit (not shown), an AD (analog-to-digital) conversion unit, or the like.

Hereinafter, the operation of heart failure degree-of-exacerbation determination system 10A will be specifically described.

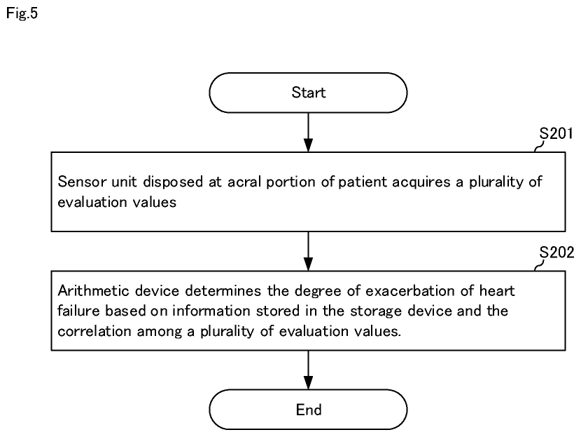

FIG. 5 is a flowchart for explaining the heart failure degree-of-exacerbation determination in heart failure degree-of-exacerbation determination system 10A shown in FIG. 4. Hereinafter, the heart failure degree-of-exacerbation determination method will be described with reference to FIGS. 2, 4, and 5.

First, in step S201, sensor unit 13 disposed at acral portion 2 of patient 1 acquires a plurality of evaluation values 111 (111A and 111B). The acquired evaluation value 111 is automatically stored in storage device 11.

Evaluation value 111 is an evaluation value related to heart failure and includes either a degree of congestion, a degree of hypoperfusion, a pulse wave shape, a cardiac output, a heart rate, and a respiratory rate. Here, sensor 131 acquires evaluation value 111A, and sensor 132 acquires evaluation value 111B. Incidentally, one sensor may acquire a plurality of evaluation values.

Subsequently, in step S202, heart failure degree-of-exacerbation determination means 121 determines the degree-of-exacerbation of heart failure based on information 112 and the evaluation value 111 stored in storage device 11.

Information 112 may be acquired by a sensor for measuring the patient or the surrounding environment and may be automatically stored in storage device 11, or may be stored in storage device 11 by the patient himself/herself, a person related to the patient, or a health care worker via the input device.

Heart failure degree-of-exacerbation determination means 121 determines the degree-of-exacerbation of heart failure from evaluation value 111 based on history information 113, patient information 114, and environmental information 115.

According to the heart failure degree-of-exacerbation determination system of the present embodiment, in addition to the effects described in the first embodiment, evaluation value 111 obtained by sensor unit 13 disposed at acral portion 2 of patient 1 is automatically stored in storage device 11, heart failure degree-of-exacerbation determination means 121 determines the degree-of-exacerbation of heart failure. Thus, it is possible to improve the real-time and reliability of the heart failure degree-of-exacerbation determination.

### Third Embodiment

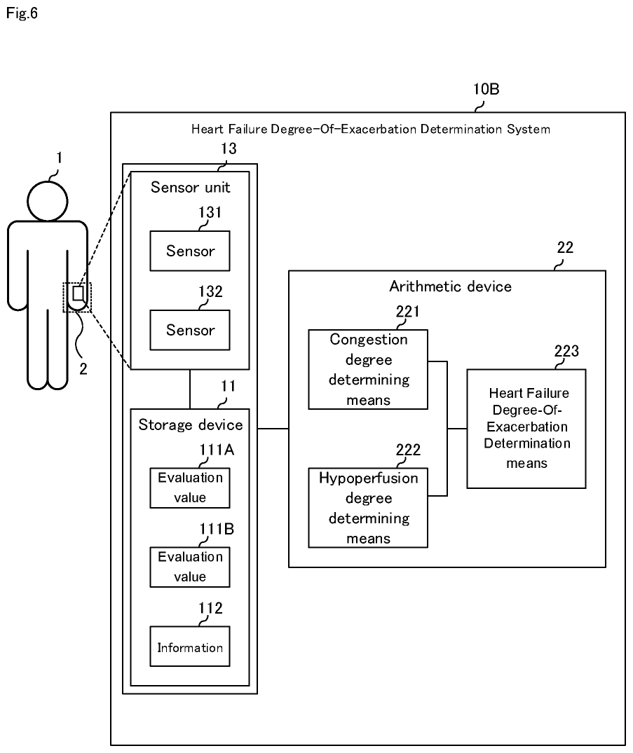

FIG. 6 is a block diagram illustrating a third embodiment of the heart failure degree-of-exacerbation determination system of the present invention. As shown in FIG. 6, although the heart failure degree-of-exacerbation determination system according to the third embodiment of the present invention has the same configuration as the first and the second embodiment, it further includes congestion degree determining means 221 and hypoperfusion degree determining means 222 in arithmetic device 22. Here, mainly configuration different from the first and the second embodiment will be described, a description of the same configuration will be omitted.

Heart failure degree-of-exacerbation determination system 10B according to a third embodiment of the present invention includes storage device 11 and arithmetic device 22 as shown in FIG. 6. Similar to the second embodiment, it may include sensor unit 13, also may not include sensor unit 13.

Arithmetic device 22 is a computer device that operates in accordance with a program, a CPU, or the like. According to the program, congestion degree determining means 221, hypoperfusion degree determining means 222, and heart failure degree-of-exacerbation determination means 223 are activated to determine the degree-of-exacerbation of heart failure. The program may be provided via a communications network or may be provided by a computer-readable recording medium. The storage medium may be storage device 11 or may be another device (not shown).

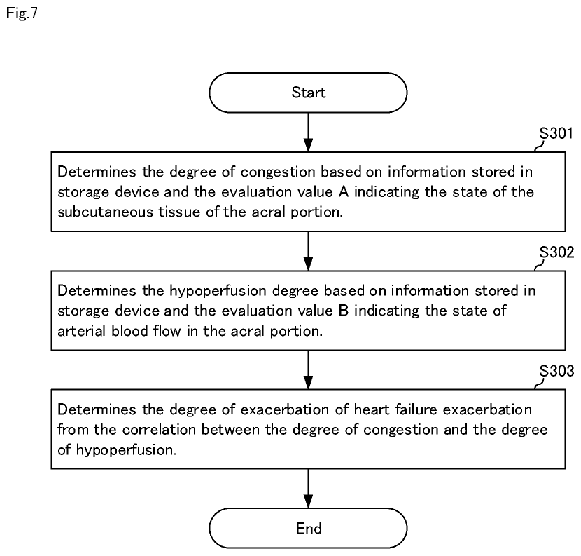

Hereinafter, the operation of heart failure degree-of-exacerbation determination system 10B will be specifically described. FIG. 7 is a flowchart for explaining a heart failure degree-of-exacerbation determination method in heart failure degree-of-exacerbation determination system 10B shown in FIG. 6. Hereinafter, the heart failure degree-of-exacerbation determination method will be described with reference to FIGS. 2, 6, and 7.

First, in step S301, congestion degree determining means 221 determines the degree of congestion based on information 112 stored in storage device 11 and evaluation value A indicating the state of the subcutaneous tissue of the acral portion.

Information 112 may be acquired by a sensor for measuring the patient or the surrounding environment and may be automatically stored in storage device 11, or may be stored in storage device 11 by the patient himself/herself, a person related to the patient, or a health care worker via the input device.

Evaluation value 111 may be acquired by non-invasive type sensor unit 13 disposed at acral portion 2 of patient 1 and may be automatically stored in storage device 11, or the values acquired by sensor unit 13 by the patient himself/herself, a person related to the patient, or a health care worker via the input device or the values determined by a physician or the like may be stored in storage device 11.

The condition of the subcutaneous tissue includes any of skin moisture content, skin elasticity, venous vessel width, and skin color. Congestion is a state in which the blood flow of the vein is stagnated and increased, accompanying phenomena such as an increase in the amount of hypodermic moisture content, a decrease in skin elasticity, darkening of skin color, and expansion of vein vessel width. Therefore, the state of the subcutaneous tissue and congestion are correlated.

With respect to the skin moisture content, that is, the water content of the subcutaneous tissue, for example, evaluation value A1 is acquired using a capacitance sensor. Since the permittivity of water is high for other materials, when an electric field is generated in the skin and electrostatic capacitance is measured, electrostatic capacitance increases when water is contained in the skin. Further, for example, evaluation value A2 regarding the skin moisture is acquired content using a near-infrared light source and a near-infrared light sensor. Water has characteristic absorption spectra in the wavelength region of near infrared light, and has absorption peaks around 1460 nm and 1920 nm wavelength. If the effect of absorbance by components other than water is excluded, the absorbance increases when the skin contains a large amount of moisture. For example, light is irradiated with a near-infrared light LED, the absorbance obtained by InGaAs (indium-gallium-arsenide) near-infrared light sensor is the evaluated value A2.

Regarding skin elasticity, for example, evaluation value A3 regarding skin elasticity is acquired using an aspirator and a displacement sensor. If skin elasticity is reduced due to congestion, the time to return to the original state after aspirating the skin is delayed. The time or speed to return to the original state is set as evaluation value A3. Further, for example, evaluation value A4 regarding the elasticity of the skin is acquired using the pusher and the displacement sensor. If skin elasticity is reduced due to congestion, the time to return to the original state after the skin is pressed is delayed.

For skin color, for example, evaluation value A5 regarding skin color is acquired using a visible light source and a spectral sensor. For example, an absorption spectrum of the skin obtained by using a white LED and a dispersive spectrometer are set as an evaluation value A5.

Regarding the venous blood vessel width, for example, evaluation value A6 regarding the venous blood vessel width in the vicinity of the dermis is acquired by using a visible light-near infrared light source and an image sensor. An image of a finger is acquired by using an LED and a camera in the vicinity of 700 nm of red light, and a portion which does not change with time among portions having a high absorbance due to the influence of hemoglobin in the blood and darkening as an image is used as a vein, and its width is set as evaluation value A6.

Thus, congestion degree determining means 221 determines the degree of congestion based on acquired evaluation values Ai (i=1 to 6), information 112 stored in the storage device, that is, history information 113, patient information 114, and environmental information 115. Specifically, as shown in Equation 1, based on information 112 in storage device 11, the congestion degree Cg is determined by the transform fi (Ai) and the weighting ui of one or more evaluation values Ai.

[Equation 1]

Cg=Σᵢ₋₁ⁿ(uᵢfᵢ(Aᵢ))/n (Equation 1)

Where ui satisfies Equation 2.

[Equation 2]

Σᵢ₌₁ⁿuᵢ=n (Equation 2)

Here, as the “evaluation value A2=relative absorbance at a wavelength of 1920 nm”, the case of not using the evaluation value other than A2, assigning the congestion degree Cg to 1 to 5. As an example, the transform fi(Ai) and the weighting ui are given by Equations 3 and 4.

$$\begin{matrix}[ Equation 3 ] & \\ {f}_{i} ( {A}_{i} ) = \{ \begin{matrix}5 & ( {A}_{i} \geq 0.96 ) \\ ⌊ 50 {A}_{i} - 43 ⌋ & ( 0.9 \leq {A}_{i} < 0.96 ) \\ 1 & ( {A}_{i} < 0.9 )\end{matrix} & ( Equation 3 ) \\ [ Equation 4 ] & \\ {u}_{i} - \{ \begin{matrix}n ( i = 2 ) \\ 0 ( i \neq 2 )\end{matrix} & ( Equation 4 )\end{matrix}$$

When relative absorbance A2 is 0.915, the congestion degree Cg is determined to be 2 based on Equations 1-4. Incidentally, Equation 3, Equation 4 based on information 112, are appropriately set.

Next, in step S302, hypoperfusion degree determining means 222 determines the hypoperfusion degree based on information 112 stored in storage device 11 and evaluation value B indicating the state of arterial blood flow in the acral portion. The states of arterial blood flow include either body temperature, amount of blood flow, skin color, or arterial vessel width. Hypoperfusion is a state in which blood is not sufficiently spread to the periphery due to the deterioration of the function of the heart, and the phenomenon such as the lowering of the body temperature in the peripheral part, lowering of amount of the arterial blood flow in the peripheral part, the blanching of the skin color, and the reduction of the arterial blood vessel width occurs.

With respect to body temperature, for example, by placing a thermistor in the peripheral portion, evaluation value B1 regarding the body temperature of the peripheral portion is acquired.

With respect to amount of blood flow, for example, ultrasound sensors (ultrasound transmitters and receivers) are used to obtain evaluation value B2 regarding the amount of blood flow in the peripheral portion. Further, for example, to obtain evaluation value B3 relating to amount of blood flow of the peripheral portion is acquired using a laser sensor (laser light source and the light receiver).

With respect to skin color, for example, evaluation value B4 regarding the extinction spectrum of the skin is acquired using a visible light source and a spectral sensor. For example, using a white LED and a dispersive spectrometer, the absorption spectrum of the acquired skin is evaluation value B4.

With respect to arterial vessel width, for example, evaluation value B5 regarding the vessel width of the artery near the dermis is acquired using a near infrared light source and an image sensor.

Thus, hypoperfusion degree determining means 222 assigns a hypoperfusion degree to, for example, 1 to 5, based on acquired evaluation value Bi (i=1 to 5), information 112 stored in storage device 11, i.e., history information 113, patient information 114 and environmental information 115. Specifically, by the conversion gi (Bi) of one or more evaluation values Bi and weighting vi based on the information in storage device 11, the hypoperfusion degree Hp, is determined.

[Equation 5]

Hp=Σᵢ₌₁ⁿ(vᵢ₀ᵢ(Rᵢ))/n (Equation 5)

Where vi satisfies Equation 6.

[Equation 6]

Σᵢ₌₁ⁿvᵢ=n (Equation 6)

As an example, as “evaluation value B1=body temperature (° C.) of the hand in an environment of 20° C. temperature”, the case of not using the evaluation value other than B 1, assigning a hypoperfusion degree to, for example, 1 to 5. As an example, the transform gi(Bi) and the weighting vi are given by Equations 7 and 8.

$$\begin{matrix}[ Equation 7 ] & \\ {g}_{1} ( {B}_{1} ) = \{ \begin{matrix}5 & ( {B}_{1} \geq 32 ) \\ ⌊ {B}_{1} - 27 ⌋ & ( 28 \leq {B}_{1} < 32 ) \\ 1 & ( {B}_{1} < 28 )\end{matrix} & ( Equation 7 ) \\ [ Equation 8 ] & \\ {v}_{i} = \{ \begin{matrix}n ( i = 1 ) \\ 0 ( i \neq 1 )\end{matrix} & ( Equation 8 )\end{matrix}$$

When body temperature B1 is 30.2° C., the hypoperfusion degree Hp is determined to be 3 based on Equations 5-8. Incidentally, Equation 7, Equation 8 based on information 112, appropriately set. Subsequently, in step S303, heart failure degree-of-exacerbation determination means 223 determines the degree-of-exacerbation of heart failure exacerbation from the correlation between the degree of congestion and the degree of hypoperfusion.

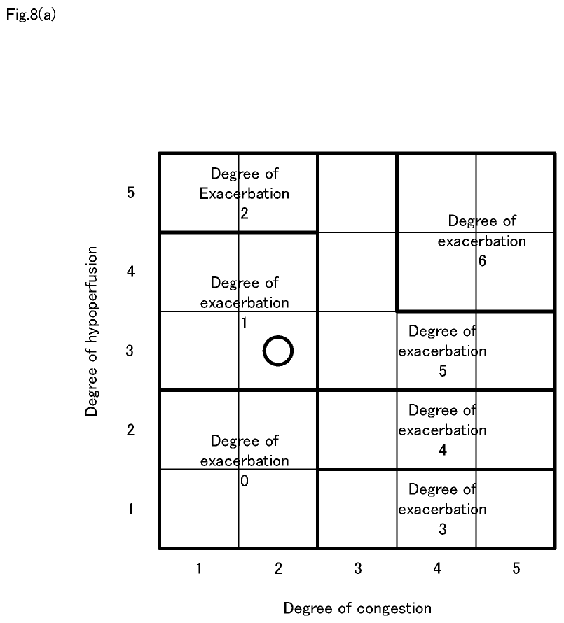

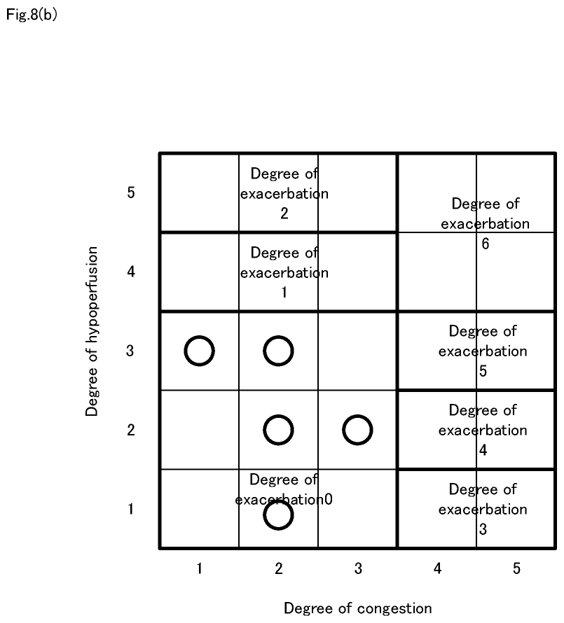

An example of the correlation between the degree of congestion and the degree of hypoperfusion is the statistical preparation of the degree-of-exacerbation map based on patient information 114 and environmental information 115. FIG. 8(a) is a diagram showing an example of a statistically created degree-of-exacerbation map. The degree-of-exacerbation map shown in FIG. 8(a) sets the degree-of-exacerbation 0-6 according to patient information 114 and environmental information 115, for example, information such as age, gender, height, weight, air temperature (minimum and maximum), and humidity of patient 1. Here, degree-of-exacerbation 0-6 indicates different classifications of degree-of-exacerbation, indicating different symptoms of heart failure for each classification of each degree-of-exacerbation. Heart failure degree-of-exacerbation determination means 223 determines the degree-of-exacerbation based on the degree of congestion and the degree of hypoperfusion determined by step S301 and step S302. Here, since degree of congestion 2 and degree of hypoperfusion 3 have been determined, heart failure degree-of-exacerbation determination means 223 is determined as the degree-of-exacerbation 1 from FIG. 8 (a). The determination result of the degree-of-exacerbation of heart failure is accumulated in storage device 11 as history information 113.

Another example of the correlation between the degree of congestion and the degree of hypoperfusion, in addition to patient information 114 and environmental information 115, is the example in which the degree-of-exacerbation map was created for each individual using historical information 113. FIG. 8 (b) is a diagram showing an example of a degree-of-exacerbation map created for each individual. Here, information as to whether or not the heart failure is controlled by the dosing or the like is always included in patient information 114. The degree-of-exacerbation map sets the degree-of-exacerbation 0-6, for example, depending on historical information 113 and patient information 114 and environmental information 115, for example, information such as patient age, gender, height, weight, air temperature (minimum and maximum), and humidity. In addition, the degree-of-exacerbation should be changed so that it becomes 0 in the usual state (the state in which the heart failure is completely controlled). For example, on a specific day, as a result of periodically obtaining the degree of congestion and the degree of hypoperfusion, as shown in FIG. 8 (b), the “congestion degree 1, hypoperfusion degree 3”, “congestion degree 2, hypoperfusion degree 1”, “congestion degree 2, hypoperfusion degree 2”, “congestion degree 2, hypoperfusion degree 3”, “congestion degree 3, hypoperfusion degree 2” when it was, degree-of-exacerbation map is appropriately changed so that the range is the degree-of-exacerbation 0 in accordance with history information 113. Thereafter, heart failure degree-of-exacerbation determination means 223 judges the degree-of-exacerbation based on the modified degree-of-exacerbation map and the degree of congestion and the degree of hypoperfusion. Here, since congestion degree 2 and the hypoperfusion degree 3 was determined, the heart failure degree-of-exacerbation determination means 223 determines the deterioration degree 0 from FIG. 8 (b). The degree of congestion, the degree of hypoperfusion and decision results of heart failure degree-of-exacerbation are accumulated in storage device 11 as history information 113. Incidentally, the determination of whether normal or not, there is a method such as determination by the health care worker, whether or not the evaluation value at the sensor indicates a steady state.

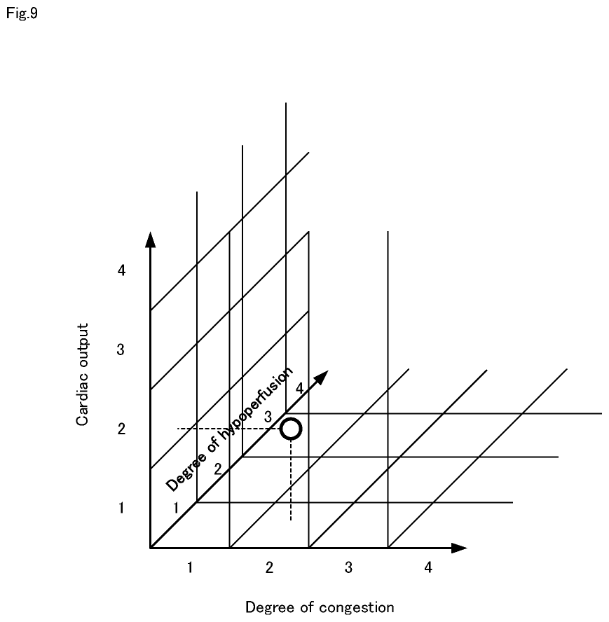

The degree-of-exacerbation map shown above is composed of two of the degree of congestion and the degree of hypoperfusion, and expressed in two dimensions, but may be constituted by two of the other evaluation values. For example, it may be a degree-of-exacerbation map composed of cardiac output and the degree of congestion. In this case, S302 determines cardiac output. In addition, the degree-of-exacerbation map may be expressed in three dimensions. FIG. 9 shows an example of a degree-of-exacerbation map expressed in three dimensions. It is composed of three types of cardiac output in addition to the degree of congestion and the degree of hypoperfusion. Heart failure degree-of-exacerbation determination means 223 assigns the degree-of-exacerbation based on the correlation of the three evaluation values, and determines the degree-of-exacerbation based on the degree of congestion, the degree of hypoperfusion, and the cardiac output.

According to the heart failure degree-of-exacerbation determination system of the present embodiment, in addition to the effects described in the first and second embodiments, based on the patient information, the environmental information, and the historical information, the degree of congestion and the degree of hypoperfusion are determined, and the degree-of-exacerbation of heart failure is determined by the correlation between the degree of congestion and the degree of hypoperfusion, and the degree-of-exacerbation map. Thus, it is possible to improve the ease of determination and reliability of the degree-of-exacerbation of heart failure deterioration.

### Fourth Embodiment

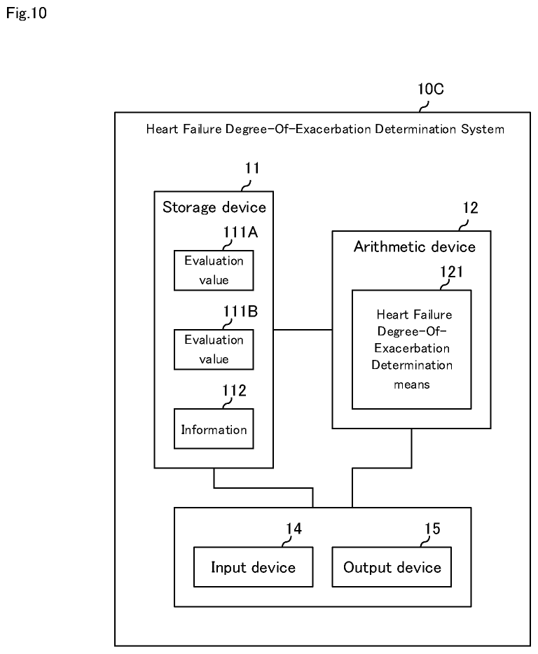

FIG. 10 is a block diagram illustrating a fourth embodiment of the heart failure degree-of-exacerbation determination system of the present invention. As shown in FIG. 10, although the heart failure degree-of-exacerbation determination system according to the fourth embodiment of the present invention has the same configuration as that of the first embodiment, it further includes input device 14 and output device 15. Here, a configuration that is different from the first to third embodiments will be mainly described, and description of the same configuration will be omitted.

Heart failure degree-of-exacerbation determination system 10C according to the fourth embodiment of the present invention includes storage device 11, arithmetic device 12, input device 14, and output device 15, as shown in FIG. 10. Similar to the second embodiment, it may include sensor unit 13.

Input device 14 is a keyboard, a voice input device, a touch panel, or the like. The value obtained by evaluation value 111 or information 112 in the sensor unit and the value determined by the health care worker or the like are stored in the storage device through input device 14 operated by the patient himself/herself, the person related to the patient, or the health care worker or the like.

Output device 15 is a display device such as a liquid crystal display or OLED (organic light emitting diodes, Organic Light-Emitting Diode) display or an audio output device such as a speaker. Through output device 15, the heart failure degree-of-exacerbation determination history is output to the health care worker or the like.

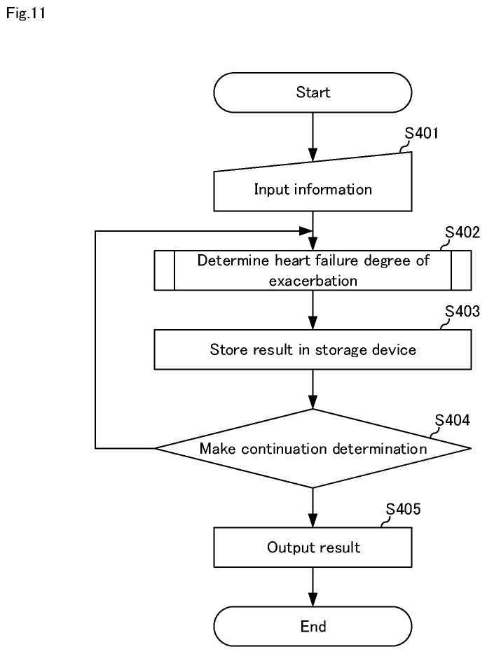

FIG. 11 is a flowchart for explaining a heart failure degree-of-exacerbation determination method in heart failure degree-of-exacerbation determination system 10C shown in FIG. 10. Hereinafter, the heart failure degree-of-exacerbation determination method will be described with reference to FIGS. 2, 3, 5, 7, 10, and 11.

First, in step S401, the patient himself/herself or a person related to the patient or a health care worker or the like operates input device 14, and evaluation value 111 and information 112 are stored in storage device 11.

In step S402, heart failure degree-of-exacerbation determination means 121 determines the degree-of-exacerbation of heart failure. The process of S402 is the same as the process of any of S101 of FIG. 3, S201, S202 of FIGS. 5, S301 to S303 of FIG. 7.

Subsequently, in step S403, heart failure degree-of-exacerbation determination means 121 stores the determination result of the degree-of-exacerbation of heart failure in storage device 11.

In step S404, the continuous determination is made. Continuous determination is a predetermined measurement time or a predetermined number of measurements, a predetermined measurement result (e.g., when it is determined that the degree-of-exacerbation is not 0), the health care worker or the system manager, etc. sets it in advance.

In step S405, the heart failure degree-of-exacerbation determination history is output to a health care worker or the like via output device 15.

According to the heart failure degree-of-exacerbation determination system of the present embodiment, in addition to the effects described in the first to third embodiments, information is appropriately input in input device 14, stored in storage device 11, continues the heart failure degree-of-exacerbation determination process continues according to the continuation determination, and the result can be output by output device 15, so that the operability of the heart failure degree-of-exacerbation determination can be improved.

### Fifth Embodiment

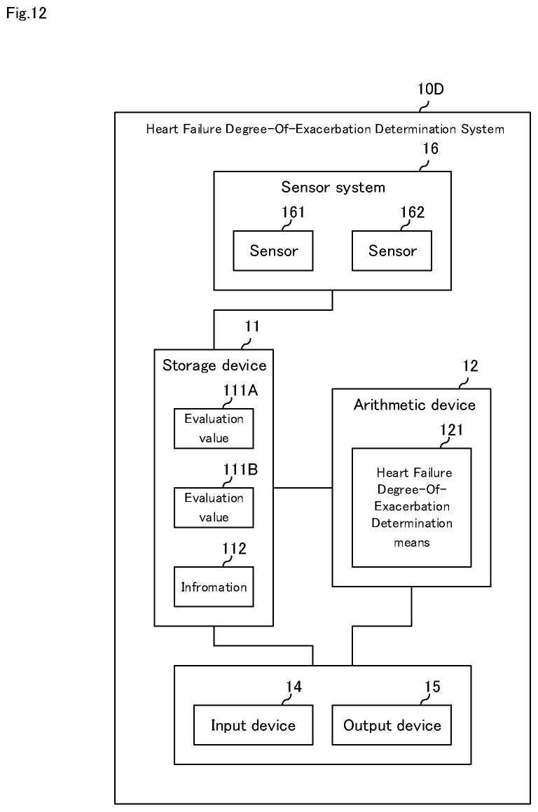

FIG. 12 is a block diagram illustrating a fifth embodiment of the heart failure degree-of-exacerbation determination system of the present invention. As shown in FIG. 12, the heart failure degree-of-exacerbation determination system according to the fifth embodiment of the present invention has the same configuration as that of the fourth embodiment, and further includes sensor system 16. Here, the configuration that is different from the first to fourth embodiments will be mainly described, and description of the same configuration will be omitted.

Heart failure degree-of-exacerbation determination system 10D according to the fifth embodiment of the present invention includes storage device 11, arithmetic device 12, input device 14, output device 15, and sensor system 16, as shown in FIG. 12. Similar to the second embodiment, it may include sensor unit 13.

Sensor system 16 acquires patient information 114 and environmental information 115. Environmental information acquisition sensors include air temperature sensors, humidity sensors, atmospheric suspended particle sensors, noise sensors, odor sensors, etc. The patient information acquisition sensors include body weight sensor, body fat rate sensor, activity amount sensor, sleep sensor, etc. Sensor system 16 may include a power supply (not shown), a communication unit, an AD (analog-to-digital) conversion unit, or the like.

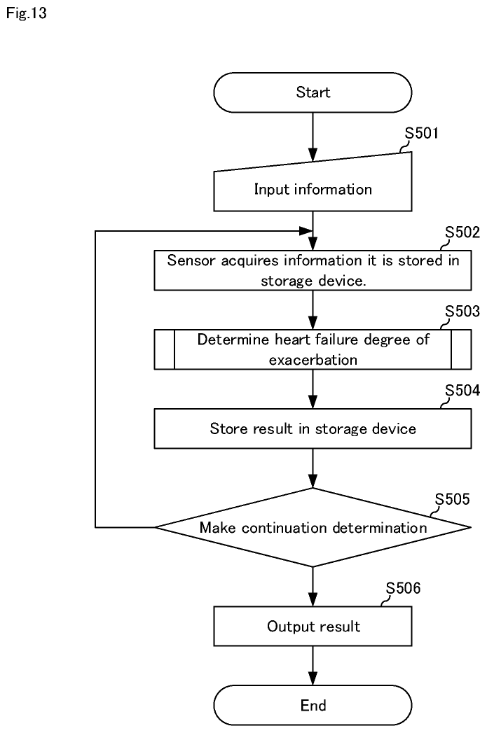

FIG. 13 is a flowchart for explaining a heart failure degree-of-exacerbation determination method in heart failure degree-of-exacerbation determination system 10D shown in FIG. 12. Next, the heart failure degree-of-exacerbation determination method will be described with reference to FIGS. 2, 3, 5, 7, 10, 12, and 13.

First, in step S501, the patient himself/herself or a person related to the patient, or a health care worker or the like operates input device 14 and evaluation value 111 and information 112 is stored in storage device 11 via input device 14.

Next, in step S502, patient information 114 and environmental information 115 are appropriately acquired in sensor system 16 and stored in storage device 11.

In step S503, heart failure degree-of-exacerbation determination means 121 determines the degree-of-exacerbation of heart failure. The process of step S503 is the same as the process of any of S101 of FIG. 3, S201, S202 of FIG. 5, S301 to S303 of FIG. 7.

Subsequently, in step S504, heart failure degree-of-exacerbation determination means 121 stores the determination result of the heart failure degree-of-exacerbation in storage device 11.

In step S505, the continuous determination is made. Continuous determination is a predetermined measurement time or a predetermined number of measurements, a predetermined measurement result (e.g., when it is determined that the degree-of-exacerbation is not 0), which the health care worker or the system manager, etc. sets in advance.

In step S506, the heart failure degree-of-exacerbation determination history is output to the health care worker or the like via output device 15.

According to the heart failure degree-of-exacerbation determination system of the present embodiment, in addition to the effects described in the first to fourth embodiments, information can be stored in storage device 11 in sensor system 16, so that the operability of the heart failure degree-of-exacerbation determination can be further improved.

### Sixth Embodiment

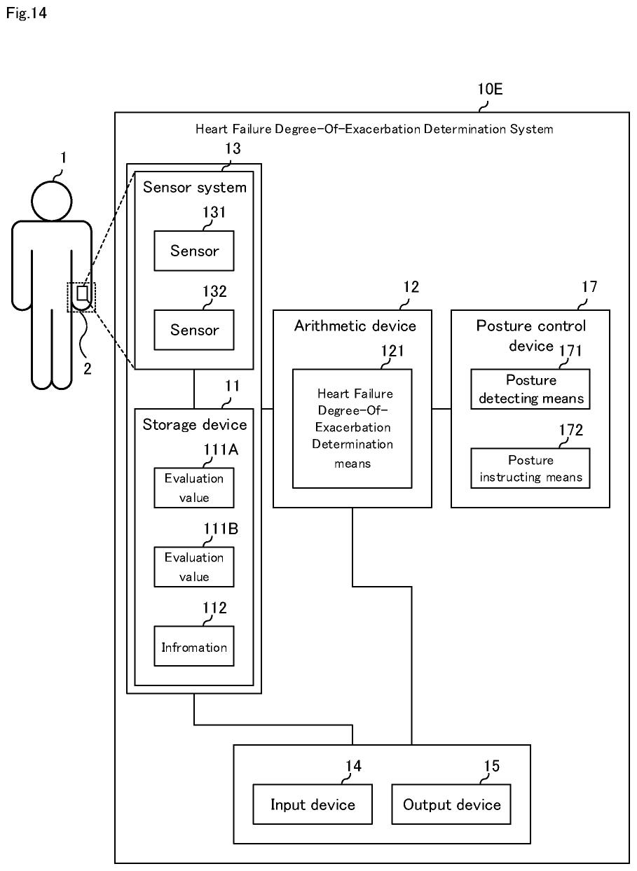

FIG. 14 is a block diagram illustrating a sixth embodiment of the heart failure degree-of-exacerbation determination system of the present invention. As shown in FIG. 14, although the heart failure degree-of-exacerbation determination system according to the sixth embodiment of the present invention has the same configuration as that of the second embodiment, it further includes posture control device 17. Here, the configuration different from the first to fifth embodiments will be mainly described, and description of the same configuration will be omitted.

Heart failure degree-of-exacerbation determination system 10E according to the sixth embodiment of the present invention includes, as shown in FIG. 14, storage device 11, arithmetic device 12, sensor unit 13, input device 14, output device 15, and posture control device 17. Similar to the fifth embodiment, sensor system 16 may also be provided.

Posture control device 17 has posture detecting means 171 and posture instructing means 172.

Posture detecting means 171 detects the posture of patient 1 and the position of acral portion 2. For example, posture control device 17 uses an image sensor to acquire image of patient 1 and surrounding images and detects the posture of patient 1 and the position of acral portion 2 based on the image.

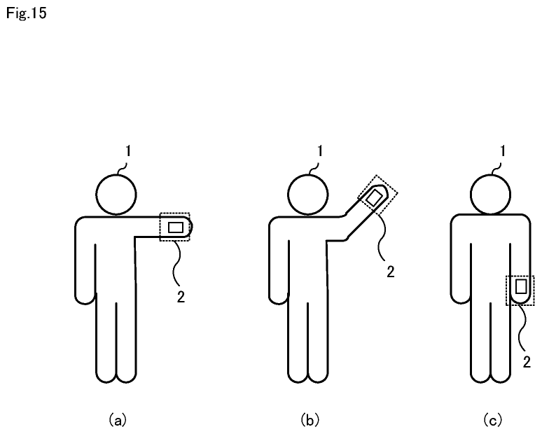

Posture instructing means 172 instructs patient 1 to the posture of patient 1 and the position of acral portion 2. Through output device 15, posture instructing means 172 specifically instructs the height of acral portion 2 in which sensor unit 13 is disposed. FIG. 15 is a diagram schematically illustrating the posture of the patient. For example, as shown in FIG. 15, for patient 1, (a) “the height of the end portion 2 is the same as the height of the heart” (b) “the height of the end portion 2 is higher than the heart” (c) “the height of the end portion 2 is lower than the heart” or the like is displayed on a liquid crystal display or audio output by a speaker.

For example, using the state change in the state of (b) and the state of (c) shown in FIG. 15 as an evaluation value, it is possible to determine the degree of congestion. Further, in the continuous determination of the degree-of-exacerbation, because continuous determination is made in a specific posture, posture detecting means 171 detects the posture, and posture instructing means 172 instructs the posture if not the desired posture. Further, it detects the posture at the time of heart failure degree-of-exacerbation determination, and stores it in storage device 11.

In the heart failure degree-of-exacerbation determination method in heart failure degree-of-exacerbation determination system 10E shown in FIG. 14, posture detecting means 171 detects the posture of patient 1, and posture instructing means 172 instructs the posture of patient 1 to execute any of the flows of FIG. 3, FIG. 5, FIG. 7, FIG. 11, and FIG. 13.

Incidentally, posture control device 17 may be configured only with one of posture detecting means 171 and posture instructing means 172.

According to the heart failure degree-of-exacerbation determination system of the present embodiment, in addition to the effects described in the first to fifth embodiments, since the posture of the patient can be controlled by posture control device 17, the reliability of the heart failure degree-of-exacerbation determination can be improved.

As described above, embodiments and application examples of the present invention have been described as exemplary examples. However, the present invention is not limited to the above-described embodiments and application examples, and various aspects that can be understood by those skilled in the art can be applied to the configuration and operation thereof.

Some or all of the above embodiments may also be described as follows, but are not limited thereto.

(Appendix 1) A heart failure degree-of-exacerbation determination system, comprising:

a storage device; and

an arithmetic device,

wherein the arithmetic device has a heart failure degree-of-exacerbation determination means that determines the degree-of-exacerbation of heart failure, based on the stored information stored in the storage device and based on the correlation between a plurality of evaluation values associated with the acral portion of the patient.

(Appendix 2) The heart failure degree-of-exacerbation determination system according to Appendix 1, wherein

the evaluation value includes any one of degree of congestion, degree of hypoperfusion, pulse wave shape, cardiac output, heart rate, and respiration rate.

(Appendix 3) The heart failure degree-of-exacerbation determination system according to Appendix 1 or 2, further comprising a sensor unit comprising one or more sensors; wherein

the sensor unit disposed at the acral portion acquires the evaluation value.

(Appendix 4) The heart failure degree-of-exacerbation determination system according to any one of Appendices 1 to 3, wherein

the arithmetic device has a congestion degree determining means that determines a degree of congestion based on the stored information and an evaluation value A indicating a state of subcutaneous tissue of the acral portion.

(Appendix 5) The heart failure degree-of-exacerbation determination system according to Appendix 4, wherein

the congestion degree determining means determines the degree of congestion by conversion and weighting based on the stored information of the evaluation value A.

(Appendix 6) The heart failure degree-of-exacerbation determination system according to any one of Appendices 1 to 5, wherein

the arithmetic device has a hypoperfusion degree determining means that determines a degree of hypoperfusion based on the stored information and an evaluation value B indicating a state of arterial blood flow at the acral portion.

(Appendix 7) The heart failure degree-of-exacerbation determination system according to Appendix 6, wherein

the hypoperfusion degree determining means determines the degree of hypoperfusion by conversion and weighting based on the stored information of the evaluation value B

(Appendix 8) The heart failure degree-of-exacerbation determination system according to Appendices 6 or 7, wherein

the heart failure degree-of-exacerbation determination means determines the degree-of-exacerbation of heart failure from the correlation between the stored information and the degree of congestion and the degree of hypoperfusion.

(Appendix 9) The heart failure degree-of-exacerbation determination system according to any one of Appendices 1 to 8, wherein

the heart failure degree-of-exacerbation determination means creates a degree-of-exacerbation map based on the stored information and determines the degree-of-exacerbation of heart failure according to a correlation between the degree-of-exacerbation map and the evaluation value.

(Appendix 10) The heart failure degree-of-exacerbation determination system according to any one of Appendices 1 to 9, wherein

the stored information includes any one of history information, patient information, and environmental information.

(Appendix 11) The heart failure degree-of-exacerbation determination system according to Appendix 10, wherein

the heart failure degree-of-exacerbation determination means statistically creates a degree-of-exacerbation map based on the patient information and the environmental information, and determines the degree-of-exacerbation of heart failure according to a correlation between the degree-of-exacerbation map and the evaluation value.

(Appendix 12) The heart failure degree-of-exacerbation determination system according to Appendix 10, wherein

the heart failure degree-of-exacerbation determination means creates a degree-of-exacerbation map for each individual based on the history information, the patient information, and the environmental information, and determines the degree-of-exacerbation of heart failure according to a correlation between the degree-of-exacerbation map and the evaluation value.

(Appendix 13) The heart failure degree-of-exacerbation determination system according to Appendix 11 or 12, wherein

the degree-of-exacerbation map is constituted by any two evaluation values of the plurality of evaluation values.

(Appendix 14) The heart failure degree-of-exacerbation determination system according to Appendix 11 or 12, wherein

the degree-of-exacerbation map is constituted by any three evaluation values of the plurality of evaluation values.

(Appendix 15) The heart failure degree-of-exacerbation determination system according to any one of Appendices 1 to 14, further comprising an input device.
(Appendix 16) The heart failure degree-of-exacerbation determination system according to any one of Appendices 11 to 14, further comprising a sensor system that acquires some or all of the patient information and the environmental information.
(Appendix 17) The heart failure degree-of-exacerbation determination system according to any one of Appendices 1 to 16, further comprising a posture control device.
(Appendix 18) A method for determining the degree-of-exacerbation of heart failure based on information and correlations between multiple evaluation values related to the patient's acral portion.

While the preferred embodiments and examples of the present invention have been presented and described in detail above, the present invention is not limited to the above embodiments and examples, and various changes and modifications may be made without departing from the gist.

This application claims priority based on Japanese Patent Application No. 2018-071734, filed Apr. 3, 2018, and incorporates all of its disclosure herein.

What is claimed is:

1. A heart failure degree-of-exacerbation determination system, comprising:
a storage device; and
an arithmetic device,
wherein the arithmetic device has a heart failure degree-of-exacerbation determination means that determines the degree-of-exacerbation of heart failure, based on the stored information stored in the storage device and based on the correlation between a plurality of evaluation values associated with the acral portion of the patient.

2. The heart failure degree-of-exacerbation determination system according to claim 1, wherein
the evaluation value includes any one of degree of congestion, degree of hypoperfusion, pulse wave shape, cardiac output, heart rate, and respiration rate.

3. The heart failure degree-of-exacerbation determination system according to claim 1, further comprising a sensor unit comprising one or more sensors; wherein
the sensor unit disposed at the acral portion acquires the evaluation value.

4. The heart failure degree-of-exacerbation determination system according to claim 1, wherein
the arithmetic device has a congestion degree determining means that determines a degree of congestion based on the stored information and an evaluation value A indicating a state of subcutaneous tissue of the acral portion.

5. The heart failure degree-of-exacerbation determination system according to claim 4, wherein
the congestion degree determining means determines the degree of congestion by conversion and weighting based on the stored information of the evaluation value A.

6. The heart failure degree-of-exacerbation determination system according to claim 1, wherein
the arithmetic device has a hypoperfusion degree determining means that determines a degree of hypoperfusion based on the stored information and an evaluation value B indicating a state of arterial blood flow at the acral portion.

7. The heart failure degree-of-exacerbation determination system according to claim 6, wherein
the hypoperfusion degree determining means determines the degree of hypoperfusion by conversion and weighting based on the stored information of the evaluation value B

8. The heart failure degree-of-exacerbation determination system according to claim 6, wherein
the heart failure degree-of-exacerbation determination means determines the degree-of-exacerbation of heart failure from the correlation between the stored information and the degree of congestion and the degree of hypoperfusion.

9. The heart failure degree-of-exacerbation determination system according to claim 1, wherein
the heart failure degree-of-exacerbation determination means creates a degree-of-exacerbation map based on the stored information and determines the degree-of-exacerbation of heart failure according to a correlation between the degree-of-exacerbation map and the evaluation value.

10. The heart failure degree-of-exacerbation determination system according to claim 1, wherein
the stored information includes any one of history information, patient information, and environmental information.

11. The heart failure degree-of-exacerbation determination system according to claim 10, wherein
the heart failure degree-of-exacerbation determination means statistically creates a degree-of-exacerbation map based on the patient information and the environmental information, and determines the degree-of-exacerbation of heart failure according to a correlation between the degree-of-exacerbation map and the evaluation value.

12. The heart failure degree-of-exacerbation determination system according to claim 10, wherein
the heart failure degree-of-exacerbation determination means creates a degree-of-exacerbation map for each individual based on the history information, the patient information, and the environmental information, and determines the degree-of-exacerbation of heart failure according to a correlation between the degree-of-exacerbation map and the evaluation value.

13. The heart failure degree-of-exacerbation determination system according to claim 11, wherein
the degree-of-exacerbation map is constituted by any two evaluation values of the plurality of evaluation values.

14. The heart failure degree-of-exacerbation determination system according to claim 11, wherein
the degree-of-exacerbation map is constituted by any three evaluation values of the plurality of evaluation values.

15. The heart failure degree-of-exacerbation determination system according to claim 1, further comprising an input device.

16. The heart failure degree-of-exacerbation determination system according to claim 11, further comprising a sensor system that acquires some or all of the patient information and the environmental information.

17. The heart failure degree-of-exacerbation determination system according to claim 1, further comprising a posture control device.

18. A method for determining the degree-of-exacerbation of heart failure based on information and correlations between multiple evaluation values related to the patient's acral portion.
